(19) United States
(12) Patent Application Publication
(10) Pub. No.: US 2002/0168718 A1
(43) Pub. Date: Nov. 14, 2002

(54) Enzyme-mediated modification of fibrin for tissue engineering

(75) Inventors: Jeffrey Hubbell, Zumikon (CH); Jason Schense, Zurich (CH); Andreas Zisch, Zurich (CH); Heike Hall, Zurich (CH)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: California Institute of Technology

(21) Appl. No.: 10/024,918

(22) Filed: Dec. 18, 2001

Related U.S. Application Data
(63) Continuation-in-part of application No. 09/057,052, filed on Apr. 8, 1998, now Pat. No. 6,331,422.
Application No. 09/057,052 is a continuation of application No. PCT/US98/06617, filed on Apr. 2, 1998.
(60) Provisional application No. 60/042,143, filed on Apr. 3, 1997.

(51) Int. Cl. C12P 21/06

(52) U.S. Cl. 435/69.1; 435/68.1; 435/69.4

(57) ABSTRACT

Matrices covalently bound to bidomain peptides or proteins, produced recombinantly or synthetically, contain a transglutaminase substrate domain and a bioactive factor. The bioactive factor is preferably a growth factor, such as VEGF, growth factors from the TGF-β superfamily, PDGF, human growth hormone, IGF, and ephrin. Particularly preferred growth factors are TGF-β1, BMP 2; VEGF₁₂₁ and PDGF AB. The matrix material is crosslinkable, and may form a gel. These compositions are useful for tissue repair and regeneration.

### CROSS-REFERENCE TO RELATED APPLICATIONS

This application is a continuation in part of U.S. Ser. No. 09/057,052, filed Apr. 8, 1998, now U.S. Pat. No. 6,331,422, which is a continuation of International Application No. PCT/US98/06617, filed Apr. 2, 1998, which claims priority to U.S. Provisional Application Serial No. 60/042,143, filed Apr. 3, 1997.

### STATEMENT REGARDING FEDERALLY SPONSORED RESEARCH

The United States Government has certain rights in this invention pursuant to Grant No: USPHS HD 31462-01A1, awarded by the National Institute of Health.

### FIELD OF THE INVENTION

This application is generally in the field of tissue repair and regeneration, and more particularly relates to matrices containing bidomain peptides or proteins. Throughout this application, various publications are referenced. The disclosures of those publications are hereby incorporated by reference into this application in their entireties.

### BACKGROUND OF THE INVENTION

Fibrin is a natural gel with several biomedical applications. Fibrin gel has been used as a sealant because of its ability to bind to many tissues and its natural role in wound healing. Some specific applications include use as a sealant for vascular graft attachment, heart valve attachment, bone positioning in fractures and tendon repair (Sierra, D. H., Journal of Biomaterials Applications, 7:309-352, 1993). Additionally, these gels have been used as drug delivery devices, and for neuronal regeneration (Williams, et al., Journal of Comparative Neurobiology, 264:284-290, 1987). Although fibrin does provide a solid support for tissue regeneration and cell ingrowth, there are few active sequences in the monomer that directly enhance these processes.

The process by which fibrinogen is polymerized into fibrin has also been characterized. Initially, a protease cleaves the dimeric fibrinogen molecule at the two symmetric sites. There are several possible proteases than can cleave fibrinogen, including thrombin, reptilase, and protease III, and each one severs the protein at a different site (Francis, et al., Blood Cells, 19:291-307, 1993). Each of these cleavage sites have been located (see FIG. 1). Once the fibrinogen is cleaved, a self-polymerization step occurs in which the fibrinogen monomers come together and form a non-covalently crosslinked polymer gel (Sierra, 1993). A schematic representation of the fibrin polymer is shown in FIG. 2. This self-assembly happens because binding sites become exposed after protease cleavage occurs. Once they are exposed, these binding sites in the center of the molecule can bind to other sites on the fibrinogen chains, which are present at the ends of the peptide chains (Stryer, L. In Biochemistry, W. H. Freeman & Company, NY, 1975). In this manner, a polymer network is formed. Factor XIIIa, a transglutaminase activated from Factor XIII by thrombin proteolysis, may then covalently crosslink the polymer network. Other transglutaminases exist and may also be involved in covalent crosslinking and grafting to the fibrin network.

Once a crosslinked fibrin gel is formed, the subsequent degradation is tightly controlled. One of the key molecules in controlling the degradation of fibrin is α2-plasmin inhibitor (Aoki, N., Progress in Cardiovascular Disease, 21:267-286, 1979). This molecule acts by crosslinking to the a chain of fibrin through the action of Factor XIIIa (Sakata, et al., Journal of Clinical Investigation, 65:290-297, 1980). By attaching itself to the gel, a high concentration of inhibitor can be localized to the gel. The inhibitor then acts by preventing the binding of plasminogen to fibrin (Aoki, et al., Thrombosis and Haemostasis, 39:22-31, 1978) and inactivating plasmin (Aoki, 1979). The α2-plasmin inhibitor contains a glutamine substrate. The exact sequence has been identified as NQEQVSPL (SEQ ID NO: 15), with the first glutamine being the active amino acid for crosslinking.

The components required for making fibrin gels can be obtained in two ways. One method is to cryoprecipitate the fibrinogen from plasma, in which Factor XIII precipitates with the fibrinogen. The proteases are purified from plasma using similar methods. Another technique is to make recombinant forms of these proteins either in culture or with transgenic animals. The advantage of this is that the purity is much higher, and the concentrations of each of these components can be controlled.

Cells interact with their environment through protein-protein, protein-oligosaccharide and protein-polysaccharide interactions at the cell surface. Extracellular matrix proteins provide a host of bioactive signals to the cell. This dense network is required to support the cells, and many proteins in the matrix have been shown to control cell adhesion, spreading, migration and differentiation (Carey, Annual Review of Physiology, 53:161-177, 1991). Some of the specific proteins that have been shown to be particularly active include laminin, vitronectin, fibronectin, fibrin, fibrinogen and collagen (Lander, Journal of Trends in Neurological Science, 12:189-195, 1989). Many studies of laminin have been conducted, and it has been shown that laminin plays a vital role in the development and regeneration of nerves in vivo and nerve cells in vitro (Williams, Neurochemical Research, 12:851-869, 1987), as well as in angiogenesis.

Some of the specific sequences that directly interact with cellular receptors and cause either adhesion, spreading or signal transduction have been identified. This means that the short active peptide sequences can be used instead of the entire protein for both in vivo and in vitro experiments.

Laminin, a large multidomain protein (Martin, Annual Review of Cellular Biology, 3:57-85, 1987), has been shown to consist of three chains with several receptor-binding domains. These receptor-binding domains include the YIGSR (SEQ ID NO:1) sequence of the laminin B1 chain (Graf, et al., Cell, 48:989-996, 1987; Kleinman, et al., Archives of Biochemistry and Biophysics, 272:39-45, 1989; and Massia, et al, J. of Biol. Chem., 268:8053-8059, 1993), LRGDN (SEQ ID NO:2) of the laminin A chain (Ignatius, et al., J. of Cell Biology, 111:709-720, 1990) and PDGSR (SEQ ID NO:3) of the laminin B1 chain (Kleimnan, et al., 1989). Several other recognition sequences for neuronal cells have also been identified. These include IKVAV (SEQ ID NO:4) of the laminin A chain (Tashiro, et al., J. of Biol. Chem., 264:16174-16182, 1989) and the sequence RNIAEIIKDI (SEQ ID NO:5) of the laminin B2 chain (Liesi, et al., FEBS Letters, 244:141-148, 1989). The receptors that bind to these specific sequences have also often been identified. A subset of cellular receptors that has shown to be responsible for much of the binding is the integrin superfamily (Rouslahti, E., J. of Clin. Investigation, 87:1-5, 1991). Integrins are protein heterodimers that consist of α and β subunits. Previous work has shown that the tripeptide RGD binds to several β1 and β3 integrins (Hynes, R. O., Cell, 69:1-25, 1992; Yamada, K. M., J. of Biol. Chem., 266:12809-12812, 1991), IKVAV (SEQ ID NO:4) binds to a 110 kDa receptor (Tashiro, et al., J. of Biol. Chem., 264:16174-16182, 1989); Luckenbill-Edds, et al., Cell Tissue Research, 279:371-377, 1995), YIGSR (SEQ ID NO:1) binds to a 67 kDa receptor (Graf, et al., 1987) and DGEA (SEQ ID NO:6), a collagen sequence, binds to the α₂, β₁ integrin (Zutter & Santaro, Amer. J. of Patholody, 137:113-120, 1990). The receptor for the RNIAEIIKDI (SEQ ID NO:5) sequence has not been reported.

Work has been done in crosslinking bioactive peptides to large carrier molecules and incorporating them within fibrin gels. By attaching the peptides to the large carrier polymers, the rate of diffusion out of the fibrin gel will be slowed down. In one series of experiments, polyacrylic acid was used as the carrier polymer and various sequences from laminin were covalently bound to them to confer neuroactivity (Herbert, C et al., J. Comp. Neurol. 365 (3): 380-391 (1996)) to the gel. The stability of such a system was poor due to a lack of covalent or high affinity binding between the fibrin and the bioactive molecule.

Very little work has been done regarding incorporating peptide sequences and other bioactive factors into fibrin gels, and even less has been done regarding covalently binding peptides directly to fibrin. However, a significant amount of energy has been spent on determining which proteins bind to fibrin via enzymatic activity and often in determining the exact sequence which binds, as well. The sequence for fibrin γ-chain crosslinking has been determined, and the exact site has been located (Doolittle, et al., Biochem. & Biophys. Res. Comm., 44:94-100, 1971). Factor XIIIa has also been shown to crosslink fibronectin to fibronectin (Barry & Mosher, J. of Biol. Chem., 264:4179-4185, 1989), as well as fibronectin to fibrin itself (Okada, et al., J. of Biol. Chem., 260:1811-1820, 1985). This enzyme also crosslinks von Willebrand factor (Hada, et al., Blood, 68:95-101, 1986), as well as α2-plasmin inhibitor (Tamaki & Aoki, J. of Biol. Chem., 257:14767-14772, 1982), to fibrin. The specific sequence that binds from α2-plasmin inhibitor has been isolated (Ichinose, et al., FEBS Letters, 153:369-371, 1983) in addition to the number of possible binding sites on the fibrinogen molecule (Sobel & Gawinowicz, J. of Biol. Chem., 271:19288-19297, 1996) for α2-plasmin inhibitor. Thus, many substrates for Factor XIIIa exist, and a number of these have been identified in detail.

It is an object of the present invention to provide matrices for tissue repair, regeneration, and remodeling having incorporated therein bioactive factors, fragments or combinations of bioactive factors which retain the activity of the bioactive factors.

### BRIEF SUMMARY OF THE INVENTION

Bidomain proteins and peptides, formed either synthetically or recombinantly, contain both a transglutaminase substrate domain, such as a Factor XIIIa substrate domain, and a bioactive factor. These proteins and peptides are covalently attached to a matrix, such as fibrin, which has a three-dimensional structure capable of supporting cell growth. In the preferred embodiment, the matrix is fibrin. The bioactive factor is preferably a growth factor, such as VEGF, growth factors from the TGF-β superfamily, PDGF, human growth hormone, IGF, and ephrin. Particularly preferred growth factors are TGF-β1, BMP 2; VEGF₁₂₁ and PDGF AB. The bioactive factor can be a peptide; a particularly preferred bioactive factor is PTH.

There are numerous applications for these matrices that are derivitized with a bioactive factor. Methods described herein incorporate an active sequence or entire factor into the gels to create gels which possess specific bioactive properties.

### BRIEF DESCRIPTION OF THE DRAWINGS

FIG. 1 is an illustration of the homodimeric structure of fibrinogen.

FIG. 2 is a schematic representation of fibrinogen.

FIG. 3 is a graph of molar excess of peptide used versus the ratio of peptide molecules to fibrinogen molecules for a series of peptide concentrations. Each curve represents the crosslinking ability of a different peptide. The Gln (SEQ ID NO:8) and Lys (SEQ ID NO:9) represent the two peptides that mimic the γ-chain of fibrinogen. Polylys is the multiple lysine peptide (SEQ ID NO:10) and pi-1 (SEQ ID NO:11) is the sequence from α2-plasmin inhibitor.

FIG. 4 is a graph of the molar ratio of peptide to fibrinogen in the reaction mixture versus the molar ratio of peptide to fibrinogen in the crosslinked fibrin gel. Each curve represents the different crosslinking abilities of the four peptides, SEQ ID NO:8, SEQ ID NO:9, SEQ ID NO:10, and SEQ ID NO: 11.

FIG. 5 is a bar graph of growth normalized against unmodified fibrin versus normalized neurite length.

### DETAILED DESCRIPTION OF THE INVENTION

I. Matrices and Bioactive Factors

A. Matrix Materials

In the preferred embodiment, the matrix is formed of proteins, most preferably proteins naturally present in the patient into which the matrix is to be implanted. The most preferred protein is fibrin. Fibrin provides a suitable three-dimensional structure for tissue growth and is a native matrix for tissue healing. Other proteins, such as collagen, polysaccharides, and glycoproteins may also be used. In some embodiments, it is also possible to use synthetic polymers which are crosslinkable by ionic or covalent binding. A recombinant form of fibrinogen can be used to form the fibrin network.

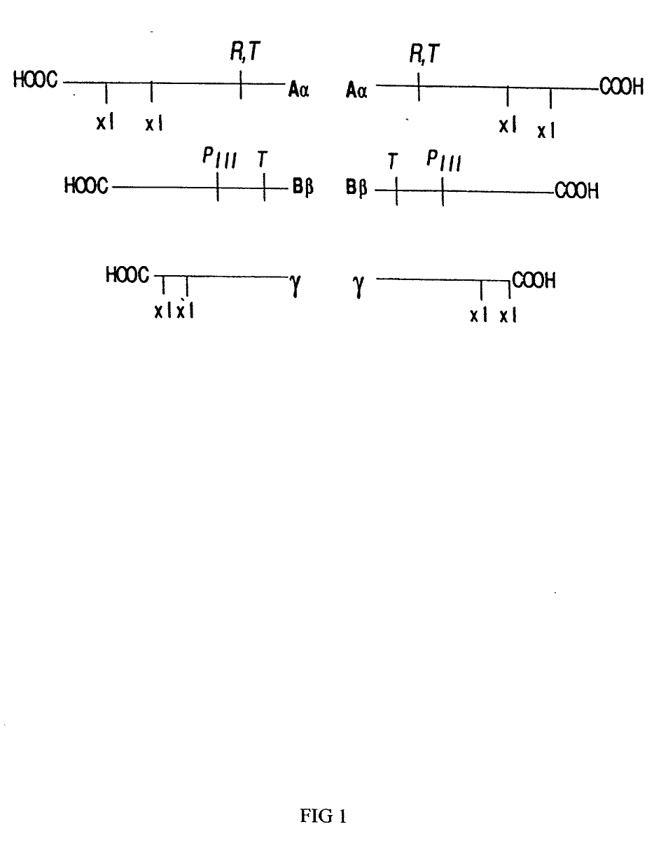

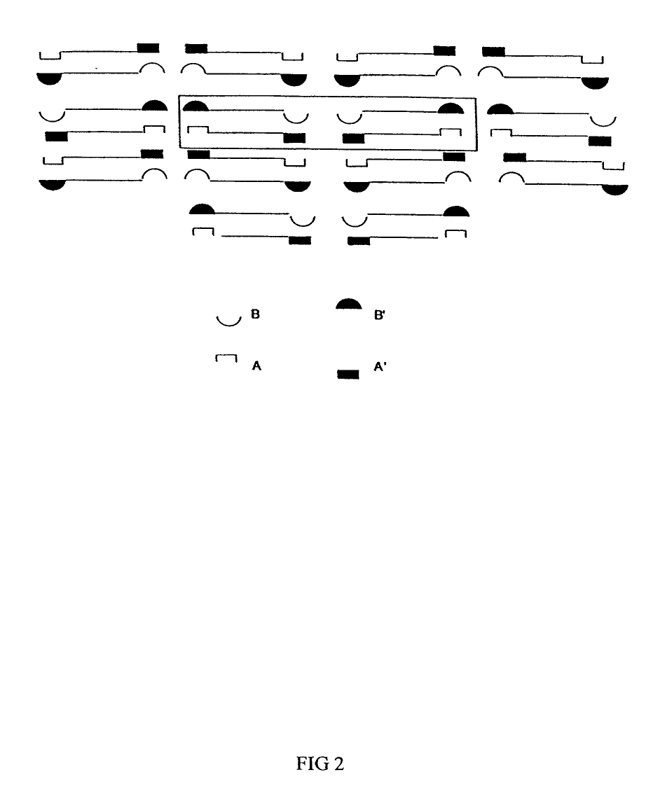

FIG. 1 is an illustration of the homodimeric structure of fibrinogen. Each symmetric half of fibrinogen is a heterotrimer of the three chains Aα, Bβ and γ. Here, the cleavage sites of the major proteases have been marked. R is for reptilase; T is for thrombin; and P III is for protease III. Additionally, some of the sites where crosslinking can occur have been marked as x1. FIG. 2 is a schematic representation of fibrinogen. The polymer is held together by the binding of sites B to B′ and A to A′. A′ and B only become available for binding after cleavage by a protease. The polymerization reaction is self-activated. A single monomer unit is boxed in the center.

The matrix material is crosslinkable, and may form a gel. A gel is a material in which a crosslinked polymer network is swollen to a finite extent by a continuous phase of an aqueous solution. The matrix material is preferably biodegradable by naturally present enzyme.

B. Bioactive Factors

Many different types of bioactive factors can be linked to the matrix. Table 1 is a list of sequence identification numbers and sequences that are referenced throughout the specification. This list includes bioactive factors and biodomain peptides (*indicates the dansyl group and the section in italics is the native sequence of the crosslinking region of fibrinogen).

TABLE 1

| Sequence ID Number | Description |
|---|---|
| SEQ ID NO: 1 | YIGSR |
| | A peptide that binds to a 67 kDa receptor |
| SEQ ID NO: 2 | LRGDN |
| | A peptide of the laminin A chain |
| SEQ ID NO: 3 | PDGSR |
| | A peptide of the laminin B1 chain |
| SEQ ID NO: 4 | IKVAV |
| | A peptide that binds to a 110 kDa receptor |
| SEQ ID NO: 5 | RNIAEIIKDI |
| | A peptide of the laminin B2 chain |
| SEQ ID NO: 6 | DGEA |
| | A collagen peptide that binds to the α₂, β₁, |
| | integrin |
| SEQ ID NO: 7 | PRRARV |
| | A sequence from fibronectin is also a heparin |
| | sulfate binding sequence |
| SEQ ID NO: 8 | *YRGDTIGEGQQHHLGG |
| | A peptide with glutamine at the transglutaminase |
| | coupling site, an active RGD sequence and a |
| | dansylated amino acid, mimics the crosslinking |
| | site in the γ chain of fibrinogen |
| SEQ ID NO: 9 | *LRGDGAKDV |
| | A peptide that mimics the lysine coupling site in |
| | the γ chain of fibrinogen with an active RGD |
| | sequence and a dansylated leucine added |
| SEQ ID NO: 10: | *LRGDKKKKG |
| | A peptide with a polysine at a random coupling |
| | site attached to an active RGD and a dansylated |
| | leucine |
| SEQ ID NO: 11 | *LNQEQVSPLRGD |
| | A peptide that mimics the crosslinking site in α2- |
| | plasmin inhibitor with an active RGD added to |
| | the carboxy terminus and a dansylated leucine to |
| | the amino terminus |
| SEQ ID NO: 12 | YRGDTIGEGQQHHLGG |
| | A peptide with glutamine at the transglutaminase |
| | coupling site in the chain of fibrinogen |
| SEQ ID NO: 13 | GAKDV |
| | A peptide that mimics the lysine coupling site in |
| | the chain of fibrinogen |
| SEQ ID NO: 14 | KKKK |
| | A peptide with a polylysine at a random coupling |
| | site |
| SEQ ID NO: 15 | NQEQVSPL |
| | A peptide that mimics the crosslinking site in α2- |
| | plasmin inhibitor (abbreviated TG) |
| SEQ ID NO: 16 | *LNQEQVSPLGYIGSR |
| | A peptide that mimics the crosslinking site in α2- |
| | plasmin inhibitor with an active YIGSR (SEQ ID |
| | NO: 1) added to the carboxy terminus and a |
| | dansylated leucine to the amino terminus |
| SEQ ID. NO: 17 | *LNQEQVSPLDDGEAG |
| | A peptide that mimics the crosslinking site in a |
| | α2-plasmin inhibitor with an active DGEA (SEQ |
| | ID NO: 6) |
| SEQ ID NO: 18 | *LNQEQVSPLRAHAVSE |
| | A peptide that mimics the crosslinking site in α2- |
| | plasmin inhibitor with an active HAV added to |
| | the carboxy terminus and a dansylated leucine to |
| | the amino terminus |
| SEQ ID NO: 19 | *LNQEQVSPRDIKVAVDG |
| | A peptide that mimics the crosslinking site in α2- |
| | plasmin inhibitor with an active IDVAV (SEQ ID |
| | NO: 4) added to the carboxy terminus and a |
| | dansylated leucine to the amino terminus |
| SEQ ID NO: 20 | *LNQEQVSPRNIAEIIKDIR |
| | A peptide that mimics the crosslinking site in α2- |
| | plasmin inhibitor with an active RNIAEIIKDI |
| | (SEQ ID NO: 5) added to the carboxy terminus |
| | and a dansylated leucine to the amino terminus |

The bioactive factor may comprise an amino acid sequence of SEQ ID NO:1, SEQ ID NO:2, SEQ ID NO:3, SEQ ID NO:4, SEQ ID NO:5, SEQ ID NO:6, a fragment thereof, a combination thereof, or a bioactive fragment of said combination.

Many different kinds of bioactive factors can be used. Longer peptide sequences, such as the truncated form of parathyroid hormone (PTH), which has a sequence that contains 34 amino acids, or L1Ig6, and proteins of molecular weights above 25,000 kDa can be attached to a transglutaminase domain and crosslinked into a fibrin matrix. The bioactive factor is preferably a growth factor, such as VEGF, growth factors from the TGF-β superfamily, PDGF, human growth hormone, IGF, and ephrin. Particularly preferred growth factors are TGF-β1, BMP 2; VEGF₁₂₁ and PDGF AB. The bioactive factor can be a peptide. A peptide can contain up to 50 amino acids. A particularly preferred bioactive factor is PTH.

Membrane-Bound Growth Factors

Favorable results have been achieved with naturally occurring membrane-bound growth factors, such as ephrin B2. In vitro and in vivo bioactivity depends on clustering the molecule and attaching it to a membrane. However, most artificial methods are both time consuming and difficult. By crosslinking growth factors, such as ephrin, into a fibrin matrix, clustering can be easily induced.

Ephrin and Eph Receptors

Ephrin ligands and their corresponding Eph receptors represent families of cell surface bound protein ligands that mediate bi-directional cell to cell signaling, and thereby guide the navigation of axons, neural stem cells, and blood vessel cells. There are fourteen known Eph receptors in mammals. Thus, Eph receptors constitute the largest subgroup of the receptor tyrosine kinases. Eph receptors are able to bind at least eight ephrin ligands, which are membrane-attached cell surface molecules, unlike the majority of ligands for receptor tyrosine kinases, which are soluble.

Genetic experiments have demonstrated that an essential function of ephrins and Eph receptors is establishing the functional topography of the developing vasculature and the nervous system. Ephrin B2 and its receptor tyrosine kinase, EphB4, are molecular markers of embryonic and venous endothelial cells, respectively. Most recent studies have indicated roles of ephrin-Eph receptor signaling in adult settings of angiogenesis, in synaptogenesis and in neural stem cell proliferation in the adult brain.

The ephrin-Eph receptor system is characterized by several unusual features. One feature of the system is that the ligands display an active signaling role. Binding between ephrin ligands and their Eph receptors on apposing cell membranes leads to reciprocal cell signaling. A second feature of this system is that membrane attachment and clustering of ephrin ligands, as multivalent affinity complexes, is critical for activity. Within their natural environment of the plasma membrane, ephrins are locally concentrated in raft membrane microdomains. The intracellular portion of ephrin proteins represents a target site for intracellular PDZ domain-containing proteins, such as GRIP. GRIP contains multiple PDZ domains and provides a scaffold for the assembly of multiple ephrin proteins in these specialized membrane compartments. A third feature of this system is that signaling is initiated by the temporary adhesion between surface-bound ephrins and Eph receptors. In the case of axons, however, the initial adhesive contact between the multivalent ephrin complex and Eph receptors on the axon finally results in a repulsion of the Eph receptor-bearing cell from the ephrin-bearing cell. As recently shown, the binding is terminated by cleavage and subsequent shedding of the ephrin extracellular domain from the plasma membrane by membrane-bound metalloproteases such as Kuzbanian that form a stable complex with ephrins.

A fourth feature of the ephrin-Eph receptor system is that as recombinant, soluble ligands, ephrin variants act as an antagonist. However, when artificially clustered, these ephrin variants exhibit growth factor-like activities for endothelial cells or mediate growth cone collapse of navigating neurons.

Display and delivery of ephrin B2 from fibrin depots, alone or in combination with blood vessel inducing factors, such as vascular endothelial growth factor (VEGF), are therapeutically useful to induce the formation of healthy vessels. Furthermore, ephrin B2 can be applied for peripheral nerve regeneration. When applied in nerve guide tubes, ephrin B2-modified fibrin helps reinnervate the target muscle. Ephrin B2-modified fibrin may be therapeutically useful in promoting the growth of large numbers of neural stem cells.

C. Bidomain Proteins and Peptides

A bidomain peptide or protein having an amino acid sequence that comprises a crosslinking domain and a bioactive factor (or peptide, protein, or fragment thereof) is provided. The bidomain peptide or protein is covalently or at least substantially covalently bound to the matrix. In the preferred embodiment, the crosslinking domain is a transglutaminase substrate domain. The transglutaminase substrate domain may be a Factor XIIIa substrate domain. This Factor XIIIa substrate domain may be further defined as comprising an amino acid sequence SEQ ID NO:12, SEQ ID NO:13, SEQ ID NO:14, or SEQ ID NO: 15, a fragments, or combinations thereof.

In a preferred embodiment the bidomain protein is a fusion protein, which comprises a transglutaminase substrate domain and a bioactive factor, such as the amino acid sequence of SEQ ID NO:16, SEQ ID NO:17, SEQ ID NO:18, SEQ ID NO:19, or SEQ ID NO:20.

The coupling between the bioactive factor and the transglutaminase substrate domain can be performed by recombinant DNA methodology or any other means. For example, a protein growth factor could be incorporated by recombinantly expressing a fusion protein comprising both a transglutaminase substrase domain and the growth factor domain.

The transglutaminase substrate domain can be a substrate for a translutaminase other than Factor XIIIa. The most preferred Factor XIIIa substrate domain has an amino acid sequence of SEQ ID NO:15 (herein referred to as “TG”). Other proteins that transglutaminase recognizes, such as fibronectin, could be coupled to the transglutaminase substrate peptide.

II. Methods for Incorporation of Bioactive Factors.

In the preferred embodiment for incorporation of a bioactive factor within the matrix, a bidomain peptide or protein is added to the matrix and the matrix is crosslinked by the native Factor XIIIa, which attaches the exogenous factors to the matrix. The bidomain peptide or protein includes one domain which is a substrate for a crosslinking enzymes, such as Factor XIIIa, and a second domain which is the bioactive peptide or protein. Factor XIIIa is a transglutaminase that is active during coagulation.

Using standard solid phase peptide synthesis, peptides with sequences that combine crosslinking sites from fibrinogen or another protein that crosslinks to fibrin gels, and active sequences, such as RGD or IKVAV (SEQ ID NO:4) were created. A dansyl group was added to the primary amine of the peptide so that the molecule could be detected when in the presence of other proteins. The peptides were syringe filtered and freeze dried to purify.

Fibrin gels were created using thrombin as the enzyme. Thrombin, calcium, dansylated peptide and Tris Buffered Saline (pH 7) were mixed to achieve the proper concentration of all components. Dialyzed fibrinogen that contains residual Factor XIIIa was added and the gels were polymerized in an incubator. The final gel concentrations for each component were 4 mg/mL of fibrinogen, 2.5 mM CA⁺⁺, 2 NIH units/mL of thrombin and various amounts of bioactive factor. The gels were then covered with Phosphate Buffered Saline, and the buffer was changed until all the free bioactive factor had diffused from the gel. The gels were then degraded with the minimal amount of plasmin necessary to achieve complete degradation.

A recombinant form of fibrinogen is described by Roy SN et al., J. Biol. Chem, 270 (40): 23761-7 (1995) and C M Redman & B. Kudryk, J. Biol. Chem, 274 (1): 554 (1999).

Methods for Quantifying the incorporation of Bioactive Factors in Matrices

The amount of bidomain peptide or protein is an amount of that falls within a physiologically relevant concentration of the particular peptide/protein selected. For a standard gel, 1 mg of fibrinogen would typically be included. Hence the concentration of fibrinogen in this standard gel may be described as about 3×10⁻⁶ mM. Using this figure as a benchmark in one example, the ratio of the amount of peptide to fibrinogen could be expressed as about 3×10⁻⁶ mM to about 24×10⁻⁶ mM.

There are several methods of measuring the incorporation of bidomain bioactive factors into fibrin matrices. One method for quantifying the presence of biodomain bioactive factors involves size exclusion chromatography. The bioactive factors are run on a gel permeation chromatography column and analyzed using a photodiode array detector. With this detector, data can be collected and analyzed at many wavelengths simultaneously. Chromatograms of each run are made at 280 nm; this signal is proportional to the total protein present. A wavelength of 205 nm can be used as well. The results are then compared to a standard curve created from degraded fibrinogen, and the total fibrin concentration is calculated.

A fluorescence detector measures the presence of peptide. The sample is excited at a wavelength of 330 nm, and the emitted energy at 530 nm is measured. This signal is proportional to the total amount of dansyl groups present. These results are compared to standards curves created for each peptide, and the ratio of peptide molecules to fibrin molecules in the gel is determined for a series of peptide concentrations.

The relative size of the peptide fragments in the gel can also be determined. If the peptide fragments are larger than the free peptide, then the peptide is directly bound to some fragment of gel and a covalent bond has been formed between the bidomain peptide and the gel.

A second method used to analyze the amount of bioactive factor in a matrix involves spectrofluorimetry. Each gel is washed several times, and the amount of peptide present in each wash is measured on a spectofluorimeter. The gels are then degraded with plasmin and then the amount of fluor present is measured. The percent of fluor in the gel compared to the washes is calculated. When the fibrinogen is dissolved, the total mass dissolved is known and is used to determine the mass of fibrinogen present in the gel. A different concentration of peptide is used in each series of studies, and curves relating the total peptide incorporated with the initial peptide used are made.

A third method for measurement of incorporation of bidomain bioactive factors into fibrin can be employed. In this method, fibrin gels are synthesized in the presence of the bidomain bioactive factor and soaked in buffer to remove free bioactive factor. The gels are then degraded with the smallest amount of plasmin necessary to degrade the fibrin gel, and run on SDS-PAGE. The gels can be analyzed with a general protein stain, using radioactivity, or with antibodies through a Western Blot.

III. Applications for the Matrices Containing Bioactive Factors

These materials may be useful in the promotion of healing and tissue regeneration, in the creation of neovascular beds for cell transplantation and in other aspects of tissue engineering.

The matrices may be used to form an implantable device having at least one surface or a portion of at least one surface that contains a bioactive factor, preferably as part of a bidomain peptide or protein, and a matrix. The implantable device may be fashioned as an artificial joint device, such as for a knee replacement.

The matrices with covalently bound bioactive factors may also take the form of a porous vascular graft, such as a scaffold for skin, bone, nerve or other cell growth. Additionally, the matrices may be used as surgical sealants or adhesives.

The matrices can also be used in methods for promoting cell growth or tissue regeneration. This method involves covalently producing a bidomain peptide or protein comprising a bioactive factor and a transglutaminase substrate domain, covalently coupling the a bidomain peptide or protein to a matrix, and exposing the matrix to cells or tissue to promote cell growth or tissue regeneration. This method may be used in conjunction with a variety of different cell types and tissue types. Such cell types include nerve cells, skin cells, and bone cells.

In some embodiments, bioactive properties found in extracellular matrix proteins and surface proteins are confined to a structurally favorable matrix that can readily be remodeled by cell-associated proteolytic activity. In some embodiments, the matrix is in the form of a gel. A transglutaminase incorporates that bidomain peptide or protein into the matrix. In addition to retaining the bioactivity of the bidomain peptide or protein, the overall structural characteristics of the matrix are maintained.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

Peptide Bound Per Molecule of Fibrinogen to Fibrin Gels.

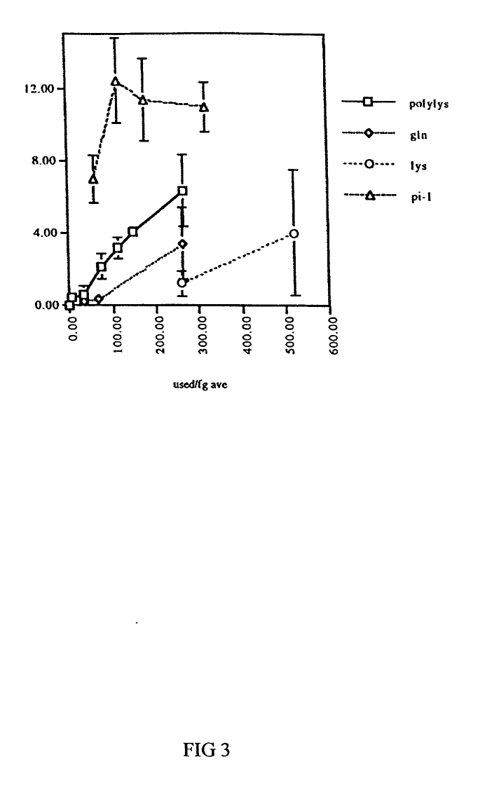

By washing peptide bound fibrin gels, degrading them with plasmin and performing size exclusion chromatography, the amount of peptide bound per molecule of fibrinogen was calculated for a series of peptide concentrations and for four separate peptide sequences. All the substrate sequences tested included RGD as an exemplary bioactive sequence. The sequences tested include two that mimic the crosslinking site in the γ chain of fibrinogen, *YRGDTIGEGQQHHLGG (SEQ ID NO:8), a peptide with glutamine at the transglutaminase coupling site, and *LRGDGAKDV (SEQ ID NO:9), a mimic of the lysine coupling site. A peptide with a polylysine at a random coupling site, *LRGDKKKKG (SEQ ID NO:10), and a sequence that mimics the crosslinking site in α2-plasmin inhibitor, *LNQEQVSPLRGD (SEQ ID NO:11) were also used. The amount of peptide covalently bound to the fibrin gels was measured while varying the initial excess of peptide for each of the four sequences. A concentration dependent curve was created (see FIG. 3), and the maximum crosslinking ratio and the molar excess needed to achieve a 1:1 ratio were calculated. The data are shown below in Table 2. Table 2 shows the amount of peptide needed to covalently bind one peptide molecule per fibrinogen molecule in a fibrin gel.

TABLE 2

| | Maximum | Molar excess |
|---|---|---|
| | Crosslinking Ratio | needed to achieve |
| Peptide Sequence | Peptide/Fibrinogen | 1:1 ratio |
| *YRGDTIGEGQQHHLGG | 1.53 | 12 |
| SEQ ID NO: 8 | | |
| *LRGDGAKDV | 0.44 | >330 |
| SEQ ID NO: 9 | | |
| *LRGDKKKKG | 1.2 | 11 |
| SEQ ID NO: 10 | | |
| *LNQEQVSPLRGD | 8.2 | 6 |
| SEQ ID NO: 11 | | |

Since a particular active sequence is usually present once in each protein, the excess of peptide required to achieve this concentration provides an interesting benchmark. The peptide that provides the greatest possible crosslinking concentration will provide the most flexibility. From the results seen in FIG. 4, the α2-plasmin inhibitor peptide (SEQ ID NO:11) is the best, since it provides the highest crosslinking concentration and the greatest crosslinking efficiency.

A collection of peptides utilizing the crosslinking sequence from α2-plasmin inhibitor have been made using active peptide sequences from the basement membrane molecules laminin and collagen (SEQ ID NO:11 and SEQ ID NOS:16-20). Eight day chicken dorsal root ganglia were polymerized inside gels that had enough peptide to achieve the highest crosslinked concentration possible (8 moles peptide/mole fibrinogen). The extension of neurites from the ganglia was measured at 24 and 48 hours. The 48 hour data is shown in FIG. 5. The average neurite length for each experimental condition was normalized against growth in unmodified fibrin. Four of the active peptides used, IKVAV (SEQ ID NO:4), RNIAEIIKDI (SEQ ID NO:5), YIGSR (SEQ ID NO:1) and RGD demonstrated statistically different neurine growth, proving that not only can different factors be attached to the fibrin gels, but they retain biologically significant activity. Soluble inhibitor experiments were completed as well, and in each trial, the neurite growth was statistically the same as unmodified fibrin. This result demonstrates that the activity is interrupted, then the presence of crosslinked peptide does not inhibit neural extension. The growth in RDG crosslinked fibrin also supports this conclusion, as the neurites are able to attain similar growth with this non-active peptide presence as is achieved in unmodified fibrin.

### Example 2

Measuring Peptide Bound Per Molecule of Fibrinogen Using Spectrofluorimetry.

This example is provided demonstrates the covalent attachment of a bioactive factor to a peptide matrix, the amount of the bioactive factor, such as a peptide, being quantitatively determinable.

Using the spectrofluorimetry method (second method) described above, the amount of peptide bound per molecule of fibrinogen was calculated for a series of peptide concentrations and for four separate peptide sequences. The sequences tested include two that mimic the crosslinking site in the y chain of fibrinogen, *YRGDTIGEGQQHHLGG (SEQ ID NO: 8), a peptide with glutamine at the transglutaminase coupling site, and *LRGDGAKDV(SEQ ID NO: 9), a mimic of the lysine coupling site. Additionally, a peptide with a polylysine at a random coupling site, *LRGDKKKKG (SEQ ID NO: 10), and a sequence that mimics the crosslinking site in α2-plasmin inhibitor, *LNQEQVSPLRGD (SEQ ID NO: 11) were also tested.

The coupling of each peptide used was measured by determining the excess moles of peptide needed to covalently bind one peptide to each fibrinogen molecule present. From the results graphically depicted in FIG. 3, the plasmin inhibitor peptide (pi-1) is has the highest coupling rate; the peptide with the sequence of multiple lysines (polylys) has the second highest coupling rate; while the two γ chain peptides (gln and lys) follow.

Table 3 lists the amount of peptide needed to covalently bind one peptide molecule to one fibrinogen molecule in a fibrin gel.

TABLE 3

| | Molar excess needed to achieve 1:1 |
|---|---|
| Peptide sequence | ratio |
| *YRGDTIGEGQQHHLGG | 110 |
| SEQ ID NO: 8 | |
| *LRGDGAKDV | 220 |
| SEQ ID NO: 9 | |
| *LRGDKKKKG | 39 |
| SEQ ID NO: 10 | |
| *LNQEQVSPLRGD | ˜10 |
| SEQ ID NO: 11 | |

### Example 3

Bioactivity In Situ Ganglia Model.

Bioactivity can be quantified using cell studies based on the 8-day chicken dorsal root ganglia model. With this model, addition of neuronally active sequences to the peptide can be tested for their ability in vitro to enhance neurite extension. Ganglia were dissected from eight day old chicken embryos and fibrin gels were polymerized around them. Peptides with different active sequences were crosslinked into these gels, and unbound peptides were washed out by periodically changing the neuronal media on top of the gels. The ganglia then extended neurites in three dimensions and the projection of these neurites was captured using imaging software. This image was used to calculate the average neurite length.

Three control experiments were performed. Neurites were grown in fibrin gels without any crosslinked peptide, in fibrin gels with a non-active peptide crosslinked to the gels and in gels with active peptide crosslinked to the gels and soluble peptide present in the media as an inhibitor.

### Example 4

Nerve Regeneration and Scaffold.

This example demonstrates a tissue regenerational supportive material. In addition, the data demonstrate regeneration of nerve tissue.

A collection of peptides utilizing the crosslinking sequence from α2-plasmin inhibitor have been made using active peptide sequences from the basement membrane molecules, laminin and collagen. Eight day chicken dorsal root ganglia were polymerized inside gels that had enough peptide to achieve the highest crosslinked concentration possible (8 moles peptide/mole fibrinogen). The extension of neurites from the ganglia was measured at 24 and 48 hours. The 48 hour data is shown in FIG. 5. The average neurite length for each experimental condition was normalized against growth in unmodified fibrin. Four of the active peptides used, IKVAV (SEQ ID NO: 4), RNIAEIIKDI (SEQ ID NO: 5), YIGSR (SEQ ID NO: 1) and RGD, demonstrated statistically different neurite growth, proving that not only can different factors be attached to the fibrin gels, but they retain biologically significant activity. Soluble inhibitor experiments were completed as well, and in each trial, the neurite growth was statistically the same as unmodified fibrin. This result demonstrates that the activity of each sequence added is dependant on the physical crosslinking. Furthermore, this shows that if the neuronal activity of the attached factor is interrupted, then the presence of crosslinked peptide does not inhibit neural extension. The growth in RDG crosslinked fibrin also supports this conclusion, as the neurites are able to attain similar growth with this nonactive peptide present as is achieved in unmodified fibrin.

### Example 5

Matrices Containing Covalently Bound TG-VEGF₁₂₁.

Cloning, purification and folding of TG-VEGF₁₂₁

A BamHI to EcoRI mutant DNA fragment corresponding to TG-VEGF₁₂₁, which contains a Factor XIIIa substrate sequence NQEQVSPL (SEQ ID NO:15) (abbreviated TG) (J. C. Schense & J. A. Hubbell, Bioconjugate Chem., 10: 75-81 (1999)) at the amino terminus of mature VEGF₁₂₁, was generated by PCR-based mutagenesis using the full length cDNA of human VEGF₁₂₁ (provided by Dr. H. Weich, National Biotechnology Research Centre (GBF), Braunschweig, Germany) as a template. The sequence of the forward primer was CGCGGA TCCAATCAAGAACAAGTCAGTCCCCTTGCA CCCATGGCAGAAGGAGGA (SEQ ID NO:21) (BamHI restriction site underlined; sequence corresponding to the additional Factor XIIIa substrate motif shown in italic letters) and that of the reverse primer was GGAATTCCTCACCGCCTCGGCTTGTCACAATTTTC (SEQ ID NO:22) (EcoRI restriction site underlined). The PCR fragment was inserted as BamiHI/EcoRI digest into pGEX-4T3 (Pharmazia) and cloned in the E. coli strain XLI Blue. Clones containing the correct VEGF DNA insert were identified by sequencing. For protein expression as a histidine-tagged protein, the BamHI/EcoRI insert was subcloned into a modified pRSET T7 expression plasmid that contains the thrombin cleavage site GLVPRG (SEQ ID NO:23) between the histidine-tag sequence and the BammHI site. The resulting plasmid pRSET TG-VEGF₁₂₁ contained a VEGF₁₂₁-construct with an aminoterminal histidine-tag linked to the thrombin cleavage site linked to TG-VEGF₁₂₁. For protein expression and purification, E. coli expression hosts BL21(DE3)pLysS or AD494 (DE3)pLysS were transformed with the pRSET-TG-VEGF₁₂₁ plasmid.

The recombinant VEGF protein was isolated as a histidine-tagged fusion protein from inclusion bodies and purified by His-Bind resin chromatography using the HIS-BIND® kit (NOVAGEN®). The transformed bacteria were grown to an OD600 of 0.6 to 0.8. Protein expression was then induced with 1 mM isopropyl-β-D1-thiogalactopyranosid for 2 to 4 hr. Bacterial cells were then pelleted and frozen at −80° C. The pellet was subsequently resuspended in a tenth of the initial culture volume in lysis buffer (50 mM Tris, pH 8.0, 2 mM EDTA), followed by the addition of lysozyme at 0.1 mg/mL lysis buffer for 15 min at 37° C. (or until the suspension became viscous). Subsequently, benzonase (Merck KGaA, Darmstadt, Germany) was added at 50 units/mL suspension, and incubated for 4 hr with magnetic stirring at 4° C. until the suspension became completely fluid. Inclusion bodies were collected by centrifugation for 30 min at 21,000 g, and then solubilized overnight at 4° C. in buffer containing 20 mM Tris, pH 7.9, 6 M urea, 5 mM imidazole and 0.5 M NaC to form a mixture. The mixture was cleared by centrifugation, and the supernatant was filtered sequentially through 5 μm and 0.22 μm filter (Millipore, Bedford, Mass.) prior to affinity purification on Ni-columns. Column chromatography was performed, as recommended by the manufacturer of the HIS-BIND® kit. Eluates from the Ni-column were collected, pooled and supplemented with 2 mM DTT to fully reduce and denature the protein into the monomeric state.

Re-folding of the VEGF fusion protein to a dimeric, di-sulfide-bonded state, was performed by sequential dialysis against 20 mM Tris, pH 7.5, 4 M urea, 1 mM EDTA, followed by dialysis buffer containing only 2 M urea. Then the histidine-tag was removed by the addition of a total of 10 units of thrombin (Sigma, plasmin-free quality) for at least 12 hr at room temperature. The removal of the histidine-tag was followed and verified by SDS-PAGE (U.K. Laemmli, Nature, 227: 680-685 (1970)) and Coomassie staining. The cleaved histidine-tag and urea were removed by final dialysis against 20 mM Tris, pH 7.5, 1 mM EDTA. The protein was further concentrated using Scorex columns, and stored until used at −80° C. in 50% glycerol. Using AD494(DE3)pLysS as the expression host, a typical yield of TG-VEGF₁₂₁ of 12 mg/L bacterial culture was obtained.

125I-labeling of TG-VEGF₁₂₁

TG-VEGF₁₂₁ was labeled with 125I using lodobeads using the following procedure. 60 μg TG-VEGF₁₂₁ in 30 μL storage buffer (20 mM Tris, pH 7.5, 1 mM EDTA, 50% glycerol), 20 μL phosphate buffer (100 mM NaH2PO4, pH 6.5), 140 μH₂O, and 1 m Ci [125I]Na (Amersham Pharmacia Biotech) were added to three lodobeads (lodobeads iodination reagent, Pierce, Rockford, Ill.). The incubation was carried out for 15 min at room temperature. After 15 min, the beads were removed. Residual free 125I was removed from the 1251-TG-VEGF₁₂₁ solution by gel filtration chromatography using as resin G25 Sephadex fine. Thereafter, the 125I-TG-VEGF₁₂₁-solution was stored in aliquots at −20° C. until used.

Polymerization of fibrin matrices and crosslinking of TG-VEGF₁₂₁

Fibrinogen solutions were prepared as described previously, using plasminogen-free fibrinogen from pooled human plasma (J. C. Schense & J. A. Hubbell, Bioconjugate Chem., 10: 75-81 (1999)); this fibrinogen contains Factor XIII, the zymogen of the transglutaminase factor XIIIa, at approximately 27 μg per mg of fibrinogen (J. C. Schense, et al., Nature Biotechnology, 18: 415-419(2000)).

Fibrin matrices were formed by mixing the components to following final concentrations: 2 to 4 mg/mL fibrinogen, 2.5 mM Ca⁺⁺, and 2 NIH units/mL human thrombin (Sigma, St. Louis, Mo.). For incorporation into fibrin, TG-VEGF₁₂₁, and the labeled 125I-TG-VEGF₁₂₁ were added to the fibrinogen solutions prior to initiation of polymerization by thrombin.

In some experiments additional exogenous Factor XIII was added. The purified Factor XIII was derived from pooled human plasma (supplied by Dr. H. Redl, Ludwig Boltzmann Institute for Experimental and Clinical Traumatology, Vienna).

Quantitative analysis of incorporated VEGF protein

The rate of VEGF-incorporation into fibrin was analyzed and quantified as follows: 100 μL VEGF-modified fibrin gels, were formed by addition of 1.6×106 counts per minute (cpm) 125I-TG-VEGF₁₂₁ and 4 μg unlabeled TG-VEGF₁₂₁ to fibrinogen (final conc. 2.6 mg/mL) at the bottom of Eppendorf tubes and γ-counted. Subsequently, unbound TG-VEGF₁₂₁ protein was removed by extensive washing. For that, gels were overlaid with 1.5 mL of TBS for a total of 5 times over 48 hr with continuous rocking of the tubes in an Eppendorf shaker at room temperature. Then, by γ-counting, the remaining 125I-TG-VEGF₁₂₁ in the fibrin gels was measured. The fibrin gels were subsequently processed for a qualitative assessment of 125I-TG-VEGF₁₂₁ incorporation as described below.

Qualitative assessment of 125I-TG-VEGF₁₂₁ incorporation

The covalent conjugation of 125I-TG-VEGF₁₂₁ to fibrinogen was analysed by SDS-PAGE and autoradiography. For this analysis, fibrin gels formed and processed by the method described above were solubilized by proteolytic digest with plasmin. The gels were overlaid with a solution of 0.02 units of plasmin in 20 μL TBS and incubated overnight at 37° C. 20 μL aliquots of the degraded fibrin solution were collected and prepared for SDS-PAGE by boiling with 5 μL of 5×SDS-sample buffer. The samples were subjected to 15% SDS-PAGE, fixed and stained with Coomassie Blue. The stained gels were then dried and exposed for autoradiography.

Endothelial cell growth assays

The mitogenic activity of soluble TG-VEGF₁₂₁ was assessed as follows: human umbilical vein endothelial cells (HUVECs) (obtained from PromoCell, Heidelberg, Germany) were seeded at 5×10³ cells/well (2.5×10³ cells per cm²) of 24 well tissue culture plates pre-coated with 0.2% gelatin in PBS. Cells were cultured for 24 hr in HUVEC growth medium (PromoCell, Heidelberg, Germany). On day 2, the medium was replaced by the medium M 199 (Life Technologies) supplemented with 10% heat denatured fetal bovine serum (FBS). This medium, when used without any additional endothelial cell growth factor supplements, represented a poor medium for endothelial cell growth. HUVECs were cultured in M199/FBS containing 0 to 300 ng/mL doses of soluble TG-VEGF₁₂₁ for 3 days at 37° C. in a fully humidified atmosphere with 5% CO₂. Then the cells were harvested from the gels by trypsination and counted.

The mitogenic activity of substrate-bound, fibrin-crosslinked TG-VEGF₁₂₁ was assessed as follows. 250 μL fibrin gels containing increasing concentrations of TG-VEGF₁₂₁ were formed at the bottom of 48 well tissue culture plates. The fibrin gels were formed by the addition of 0.1 to 10 μg TG-VEGF₁₂₁/mL fibrin gel. Control gels were made of fibrin only. Subsequently, unbound TG-VEGF₁₂₁ was removed by washing with TBS. For the removal of unbound TG-VEGF₁₂₁, the gels were overlaid seven times with 950 μL TBS over 48 hr. Then HUVECs (passage 6) in endothelial cell growth medium were seeded at 0.75×103 cells/well onto the gels and cultured for 24 hr. Next, the medium was replaced with M199/10% FBS, and the cells were grown for additional 72 hr at 37° C. in a humidified atmosphere with 5% CO₂. Thus the total cell culture time was 96 hr. The cells were then fixed with 10% formalin in neutral buffered solution, followed by May-Gruenwald staining (Sigma, St. Louis, Mo.). In some experiments, the cells were cultured for 144 hr in M199/FBS (with a total culture time of 168 hr).

In each experiment, phase pictures of the centerfields of the well were taken using the 4× objective and a Zeiss Axiovert 135 microscope equipped with an digital camera. Cells were counted from printed micrographs.

Generation of a mutant VEGF protein, TG-VEGF₁₂₁, for covalent linkage intofibrin

A fibrin modification scheme was employed to covalently incorporate VEGF₁₂₁ into fibrin by the transglutaminating activity of Factor XIII (J. C. Schense & J. A. Hubbell, Bioconjugate Chem., 10: 75-81 (1999)). Through the covalent incorporation, release of VEGF₁₂₁ was dependent on the stability of fibrin itself, as only degradation of fibrin should result in VEGF release. Incorporation was accomplished by generating through recombinant DNA methodology a novel molecule, TG-VEGF₁₂₁, containing the Factor XIIIa substrate sequence NQEQVSPL (SEQ ID NO:15) from μ2-plasmin inhibitor added to the aminoterminus of mature VEGF₁₂₁. TG-VEGF₁₂₁ was expressed and purified as Histidine-tagged fusion protein in E. coli and purified by Ni-column chromatography under denaturing conditions. The purity of the E. coli derived Histidine-tagged TG-VEGF₁₂₁ was assessed by SDS-PAGE, followed by Coomassie staining. The Histidine-tag was subsequently removed by digestion with thrombin, leading to TG-VEGF₁₂₁ with an estimated molecular weight of 16 kDa in its reduced, denatured and therefore monomeric form.

Dimerization of VEGF proteins is a functional requirement. Therefore, to permit dimerization, the VEGF solution was sequentially and extensively dialysed to remove denaturing compounds. Hereby monomeric TG-VEGF₁₂₁ was successfully folded to its dimeric form with an apparent molecular weight of ca. 32 kDa in SDS-PAGE under non-reducing conditions, consistent with the behaviour of non-modified VEGF proteins described in the literature (R. Birkenhäger, et al, Biochem. J., 316: 703-707 (1996)).

Soluble TG-VEGF₁₂₁ retains its mitogenic activity for endothelial cells

The mitogenic activity of TG-VEGF₁₂₁ was assessed in proliferation assays with human umbilical vein endothelial cells. In soluble form, TG-VEGF₁₂₁ retained its mitogenic activity for endothelial cells and stimulated endothelial cell proliferation by 47% at an optimal dose of 100 ng/mL (3.1 nM). The dose response effect appeared to be biphasic, with substantial enhancement of proliferation at 100 ng/mL, but less enhancement at higher doses. Both dose and the extent of the enhancement of cell proliferation by TG-VEGF₁₂₁ compared well the published activity of unmodified, E. coli-derived VEGF₁₂₁ (B. A. Keyt et al., J. Biol. Chem.; 271: 7788-7795 (1996)). This indicated that despite the addition of the aminoterminal factor XIII substrate sequence, the structural integrity and the active conformation of VEGF₁₂₁ remained preserved. Also, the extra domain did not seem to interfere with the binding between VEGF₁₂₁ and its endothelial cell surface receptor.

Factor XIIIa mediated incorporation of TG-VEGF₁₂₁ into fibrin Using radiolabeled 125I-TG-VEGF₁₂₁, the Factor XIII-mediated crosslinking of TG-VEGF₁₂₁ into fibrin was demonstrated. The crosslinking enzyme, Factor XIII, is a ‘contaminant’ of every fibrinogen preparation. In the present study, we used a commercial human fibrinogen preparation that contained 27 μg of Factor XIII per mg of fibrinogen (J. C. Schense et al., Nature Biotechnology, 18: 415-419 (2000)). γ-counting of gels before and after extensive washing revealed incorporation of 57% of 125I-TG-VEGF₁₂₁.

In a different experimental series, with the same batch of fibrinogen, the rate of incorporation of TG-VEGF₁₂₁ consistently ranged between 50 and 60%. Notably, this incorporation could be further enhanced by the exogenously added purified Factor XIII. Whereas the incorporation rate was only marginally affected by low concentrations (0.01, 0.05, 0.1 units/mL gel) of exogenously added Factor XIII, it was significantly raised upon addition of 1 U/mL Factor XIII (74.7%) and reached almost quantitative levels (95.6%) at 10 units of Factor XIII per mL of fibrin gel. Hence, due to the incomplete incorporation in the absence of exogenous Factor XIII, and without washing of the gels, the resulting fibrin matrices contained a combination of free and matrix-bound VEGF protein that could display both chemotactic and haptotactic features for endothelial cell recruitment.

The crosslinking of 125I-TG-VEGF₁₂₁ to fibrinogen chains was further assessed by SDS-PAGE and autoradiography.

The amount of TG-VEGF₁₂₁ in the fibrin matrix can be readily controlled by the amount of TG-VEGF₁₂₁ added to the reaction mix. This was demonstrated by measuring the incorporation of 125I-TG-VEGF₁₂₁ into fibrin upon addition of increasing doses of radiolabeled 125I-TG-VEGF₁₂₁ mixed in fixed ratio with increasing doses of the unlabeled TG-VEGF₁₂₁s. Over the whole concentration range tested (1 to 120 μg TG-VEGF₁₂₁/mL fibrin gel), the amount of incorporated TG-VEGF₁₂₁ appeared to be linearly dependent on the amount of added TG-VEGF₁₂₁. No saturation of incorporation was observed. Subsequent qualitative analysis of these fibrin gels by SDS-PAGE and autoradiography confirmed the linear, non-saturated incorporation of TG-VEGF₁₂₁ into fibrin within this concentration range.

Fibrin-bound VEGF₁₂₁ promotes endothelial cell growth

The preservation of VEGF₁₂₁ activity upon crosslinking into fibrin was assessed in a two-dimensional endothelial cell growth assay. Fibrin matrices were formed as 250 μL gels at the bottom of 48 well plates and covalently modified with VEGF₁₂₁ by the addition of 0.1 to 10 μg/mL of TG-VEGF₁₂₁ into the reaction mix. The gels were subsequently freed of any unbound, unreacted TG-VEGF₁₂₁ by extensive washing with neutral buffer solution. HUVEC were seeded on top of these fibringels at low density (0.95×10³ cells/cm²) and cultured for a total of 4 days before cell number analysis. An increasing load of covalently conjugated VEGF₁₂₁ in fibrin resulted in substantially enhanced endothelial cell numbers, up to maximum value of 239±25.7, which was set as 100%. Doses of TG-VEGF₁₂₁ higher than 1 μg/mL resulted in no further, but rather less enhancement of endothelialization. Remarkably, the promotion of endothelial cell growth by substrate-bound TG-VEGF₁₂₁ was substantially higher compared to TG-VEGF₁₂₁ in soluble form (139% versus 47%).

The positive effect of VEGF₁₂₁ incorporation into fibrin for endothelial cell growth can be particularly appreciated by inspection of micrographs taken of HUVECs cultured on VEGF₁₂₁-loaded fibrin gels for a 7 day period. Here, the differences in cell densities on fibrin modified with TG-VEGF₁₂₁ compared to the cell density on non-modified fibrin became strongly apparent.

Overall, these results demonstrate that covalently linked, substrate-bound VEGF₁₂₁ remained an active and efficient mitogen for endothelial cell growth, which is one of the key characteristics of VEGF action in endothelial cell biology.

### Example 6

Matrices Containing Covalently Bound TGL1Ig6.

Cloning, purification andfolding of TGL1Ig6

The cDNA encoding the 6th Ig-like domain (residues 516-604) of the cell adhesion molecule L1 was generated by PCR from the cDNA comprising the entire extracellular domain of L1 (P11627, Swiss Prot.) (Hall et al., J. Neurochem. 75: 336-346 (2000)). Using the primers IS (5′-CCCGGATCCC GCAGCGCAATTGAGAA-3′) (SEQ ID NO:24) and 2AS (5′-CCCGAATTCTTATTACTGTGCCCTGCTCTCC AACC-3′) (SEQ ID NO:25) produced the soluble form of L1Ig6. Sense- and anti-sense primers were designed to introduce a BamH1 site at the 5′; and two stop codons followed by an EcoR1 site at the 3′ end. A hybrid form between L1g6 and the transglutaminase Factor XIIIa substrate derived from α2-plasmin inhibitor (referred to as TGL1Ig6) was produced (Schense and Hubbell, Bioconj. Chem. 10: 75-81 (1999)). The Factor XIIIa transglutaminase substrate, NQEQVSPL (SEQ ID NO:15), was added at the N-terminus of L1Ig6 by the primers 3S (5′-CCCGGATCCAATCAGGAACAG GTTTCTCCTTTGGAAGCAACCCAGATCACACAG-3′) (SEQ ID NO:26) and 1AS. All DNA manipulations were performed according to standard protocols (Sambrook et al., Cold Spring Harbor Laboratory Press, Cold Spring Harbor, 1989) and protein expression and purification was performed as described before (Blaess et al., J. Neurochem. 71: 2615-2625 (1998)).

Sequence of L1Ig6

The sixth Ig-like domain (residues 516-604 of SEQ ID NO:27) of the cell adhesion molecule L1 (P11627, Swiss Prot.) is described below.

```
516
|
ATQIT QGPRSAIEKK GARVTFTCQA SFDPSLQASI TWRGDGRDLQ
ERGDSDKYFI EDGKLVIQSL DYSDQGNYSC VASTELDEVE SRAQ
                                                                                              |
                                                                                          604
```

Production of native and modifiedfibrin matrices

Human fibrinogen was solubilized according to standard protocols (Nehls & Herrmann, Microvasc. Res. 51: 347-364 (1996)) and fibrin matrices were formed by making a master mix of fibrinogen (2 mg/mL), recombinant protein soluble, L1Ig6 or TGL1Ig6, and TBS. Gelation was induced by adding 3.5 NHIU/mL thrombin to activate inherent Factor XIIIa. Gelation was continued for 1 hr at 37° C., and matrices were analyzed concerning their angiogenic properties.

Microcarrier bead outgrowth assayfor in vitro bioactivity

Microcarrier bead outgrowth assays were performed to culture HUVECs in three-dimensional fibrin matrices. 2 mg/mL fibrin matrices were prepared in the absence and in the presence of TGL1Ig6. In additional samples, native L1g6 was added to fibrin matrices in a soluble form. HUVECs were cultured on gelatin-coated microcarrier beads (Cytodex 3, Sigma), in a manner similar to protocols described by Nehls & Herrmann (Microvasc. Res. 51: 347-364 (1996)). Microcarrier beads were harvested, included into modified or native fibrin matrices, and maintained under low serum conditions (2%) in the absence of additional growth factors. Outgrowth of HUVEC processes from the microcarrier beads into the surrounding matrices was monitored daily.

In vivo cell infiltration assay

Medical grade silicon tubes with a 1 mm diameter (SILCLEAR® Tubing, Degania, Israel) were filled with native and TGL1Ig6-modified fibrin. Additional tubes were filled with full-length extracellular L1-containing fibrin matrices. The matrices were allowed to polymerize for 2 hours at 37° C., and 4 to 8 tubes of 0.5 cm length were implanted subcutaneously into BALB/c mice. The tubes were recovered and fixed after 1 and 5 days. The presence of cells was recorded by light microscoping and analyzed with image processing software.

In vitro Results

TGL1Ig6-modified fibrin matrices induced extensive morphologic changes of HUVECs. HUVECs appeared elongated and form processes on the surface of the substrate that interconnect small islets of cells. Cells also invaded into the three-dimensional fibrin network. In comparison to the positive effects with the TGL1Ig6 modified fibrin, fibrin matrices produced in the presence of soluble L1Ig6 provided a minimal substrate for HUVEC cell adhesion. Many cells remained round and did not spread nor form tubular structures. Finally, HUVECs grown on native fibrin spread very well and formed a stable monolayer without transforming into the angiogenic phenotype.

The network-like phenotype observed on L1Ig6-modified fibrin matrices was described for other substrates to be an indication of angiogenic differentiation in vitro (see Pepper et al., Enzyme Protein 49: 138-162 (1996)).

HUVECs grown on microcarrier beads extended multi-cellular and lumen containing process into fibrin matrices modified with TGL1Ig6. In the presence of soluble L1Ig6 outgrowing processes into the fibrin were short and few in number, whereas soluble VEGF₁₆₅ promoted strong HUVEC infiltration and process extension into the matrices, similar to covalently immobilized iL1Ig6. In native fibrin matrices, HUVECs did not extend processes nor invade the matrix.

In vivo Results

When tubes were placed subcutaneously into mice, cell infiltration was observed. This cell migration had two observable characteristics. Near the ends of the tubes, a dense mass of cells penetrated into the fibrin network. However, in the center of the tube, only single cells penetrated.

Both of these types of cell infiltration were measured, and clear differences between modified and unmodified fibrin were recorded. When TGL1Ig6 modified fibrin was used, matrices promoted cell infiltration in vivo in a concentration-dependent manner, indicating the importance of L1Ig6 as a substrate for infiltrating cells. The zone of massive cellular infiltration after 1 day was similar for native fibrin and modified fibrin matrices containing 10 μg/mL covalently immobilized TGL1Ig6 (0.12±0.08 mm² and 0.1±0.04 mm², respectively). Fibrin matrices modified with 100 μg/mL TGL1Ig6 and 50 μg/mL of full-length extracellular L1 resulted in an approximately three-fold increase in cellular infiltration after 1 day (0.321±0.08 mm² and 0.303±0.03 mm²). After 5 days, the zone of massive cellular infiltration was proportional to the concentration of TGL1Ig6, zones of 0.59±0.05 mm², 1.05±0.08 mm² and 1.25±0.27 mm² were determined for 0, 10 and 100 μg/mL TGL1Ig6. Incorporation of the whole extracellular part of L1 at 50 μg/mL induced 0.689±0.15 mm² of cellular infiltration. Furthermore, beyond the front of massive cellular infiltration, the zone of single cell infiltration also increased relative to native fibrin matrices in 10 and 100 μg/mL TGL1Ig6-modified fibrin matrices (0.8±0.09 mm² and 1.08±0.18 mm², versus 0.448±0.077 mm² in fibrin). Single cell infiltration beyond the zone of massive cellular infiltration for L1-modified matrices was found to be 0.77±0.16 mm².

When covalently bound to a fibrin matrix by a suitable transglutaminase domain, such as TG, the truncated version of L1Ig6 showed favourable results and even a higher activity than the full length L1Ig6.

### Example 7

Matrices Containing Covalently Bound TGPTH.

Cloning, purification andfolding of TGPTH

Parathyroid hormone (PTH) is an 88 amino acid protein, which has shown interesting effects in vivo as a therapeutic for osteoporosis and other applications. Furthermore, the activity has been isolated to the first 34 amino acids on the amino termini of the protein, with the 34-mer peptide showing similar activity to the whole protein, and proteins of this length can be synthesized by standard solid state peptide synthesis methods.

All peptides were synthesized on solid resin using an automated peptide synthesizer using standard 9-fluorenylmethyloxycarbonyl chemistry. Peptides were purified by c18 chromatography and analyzed using reverse phase chromatrography via HPLC to determine purity as well as mass spectroscopy (MALDI) to identify the molecular weight of each product. Using this method, the following peptide (TGPTH) was synthesized:

NH₃-Asn-Gln-Glu-Gln-Val-Ser-Pro-Leu-Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val-His-Asn-Phe-COOH (SEQ ID NO:28)

In vivo Results

The activity of TGPTH for enhancing bone regeneration was tested in a Tissucol matrix in a sheep drill hole defect. Eight mm holes that are 12 mm deep were created in the proximal and distal femur and humerus of sheep. These holes were filled with an in situ polymerizing fibrin gel. Defects were left empty, filled with Tissucol or TGPTH was added to Tissucol fibrin at 400 μg/mL before polymerization. In each example in which Tissucol was used, it was diluted four fold from the standard concentration available, leading to a fibrinogen concentration of 12.5 mg/mL.

The defects were allowed to heal for eight weeks. After this healing period, the animals were sacrificed, and the bone samples were removed and analyzed by micro computerized topography (μCT). The percent of the defect volume filled with calcified bony tissue was then determined. When defects were left empty, there was no formation of calcified tissue inside the fibrin matrix. When only a fibrin gel was added, there was practically no bone healing as well. However, with the addition of 400 μg/mL of TGPTH, the level of healing increased dramatically, with the defect filled 35% with calcified bone.

### Example 8

Matrices Containing Covalently Bound TGephrin B2.

Cloning of TGephrin B2

TGephrin B2 represents a recombinant, mutant ephrin B2 protein containing an additional eight amino acid sequence motif NQEQVSPL (SEQ ID NO:15) derived from α2-plasmin inhibitor fused to the aminoterminus of the extracellular domain of chicken ephrin B2. The extracellular domain of ephrin B2 contains amino acids 28 (immediately downstream of the ephrin B2 signal peptide cleavage site) to 224. The cDNA sequence encoding TGephrin B2 in the bacterial expression plasmid pRSET (Novagen) was obtained by two rounds of PCR-based cloning. In the first cloning step, the template was cDNA of full length chick ephrin B2 (Genbank accession number AF180729; the cDNA was provided by Dr. Elena Pasquale, The Burnham Institute, La Jolla, USA). A mutated ephrin B2 extracellular domain was generated with the Factor XIIIa substrate sequence at the aminoterminus and two additional cysteines at the C-terminus and tagged for expression and purification as glutathione S-transferase fusion (GST) protein in the bacterial expression plasmid pGEX4T3 (AmershamPharmazia). The sense primer, encoding the Factor XIIIa substrate sequence and sequences immediately downstream of the signal peptide cleavage site, and also including a custom BamHI restriction site, had the following sequence CGCGGATCCAATCAAGAACAAGTCAGTCCCCTTAAGTCCATCGTTTTAG AC (SEQ ID NO:29) (BamHI restriction site underlined; sequence corresponding to the additional Factor XIIIa substrate domain shown in italic letters). The antisense primer containing sequences immediately upstream of the transmembrane domain and a custom NotI site had the following sequence AGTCACGATGCGGCCGCGCAGCATTCTGAACCCAGTATACTGGA (SEQ ID NO:30) (NotI site underlined; sequence corresponding to two additional cysteines in italic letters). The PCR product was digested with BamHI and NotI and ligated to similarly digested pGEX4T3.

Since purification of TGephrin B2 as GST-fusion protein in E. coli appeared to be impractical, a simpler TGephrin B2 variant protein was generated by PCR for expression in the bacterial expression plasmid pRSET (Novagen) using as the template the mutated GST-ephrinB2 construct in pGEX4T3. The sense primer encoding part of the Factor XIIIa substrate and a custom NdeI site that also contains the start codon ATG had the following sequence: GGAATTCCATATGAATCAAGAACAAGTCAGTCCC (SEQ ID NO:31) (NdeI site underlined). The antisense primer was prepared with the stop codon immediately following aminoacid 224 of ephrinB2 and a custom BamHI site and had the following sequence: CGCGGATCCTCATTCTGAACCCAGTATACT (SEQ ID NO:32) (BamHI site underlined). The PCR product was digested with NdeI and BamHI and ligated into the similarly digested plasmid pRSET. The resulting plasmid Prset-TGephrin B2 encodes a mutated ephrin B2 extracellular domain with the peptide motif MNQEQVSPL (SEQ ID NO:33) aminoterminal to aminoacids 28-224 of ephrin B2. pRSET -TGephrin B2 does not provide any additional sequence tags for affinity purification.

Expression, Folding and Purification from Escherichia coli

Nonglycosylated TGephrin B2 from bacterial expression was prepared as follows. Transformed E. coli hosts JM 109 or AD494 (DE3)pLysS were lysed by addition of lysozyme, and the insoluble TGephrin B2 protein was recovered as inclusion bodies after centrifugation. The insoluble pellet was washed with 4M urea in 20 mM Tris buffer at pH 8, 2 mM EDTA before solubilization and denaturation in 8 M urea, 20 mM Tris, pH 8, 2 mM EDTA, 2 mM dithiothreitol by overnight stirring at 4° C. Insoluble bacterial proteins were then removed by centrifugation.

Analysis of the extract by SDS-PAGE and Coomasie stain revealed a protein of molecular size of 25 K that represented 98% or greater of total protein. The identity of this protein as TGephrin B2 protein was verified by immunoblotting with ephrin B2-specific antibodies.

For refolding, TGephrin B2 was subsequently dialyzed sequentially against 20 mM Tris buffer, pH 7.5, 150 mM NaCl, 1 mM EDTA containing 6 M urea, followed by Tris buffer with 4 M urea, 2 M urea, 1 M urea. Final dialysis was performed against Tris buffer only. Protein aggregates were removed by centrifugation. The cleared fraction was analyzed by non-reducing SDS-PAGE and immunoblotting to reveal a major fraction of monomeric TGephrin B2 protein and minor amounts of multimeric TGephrin B2 aggregates. Subsequently, monomeric and multimeric TGephrin B2 were fractionated by Sephadex G25 gel filtration chromatography. Homogenity and identity of the fractionated monomeric respective multimeric TGephrin B2 proteins were established by SDS-PAGE and protein Coomassie staining, as well as immunoblotting with ephrin B2 specific antibodies.

Results

The truncated variant TGephrin B2 that comprises the entire extracellular domain of ephrin B2 together with an additional eight amino acid sequence motif NQEQVSPL (SEQ ID NO:15) at the aminoterminus was purified in soluble, nonglycosylated form from E. coli. Using radiolabelled TGephrin B2, its covalent incorporation into fibrin by Factor XIII was demonstrated. The functionality of fibrin-conjugated ephrin B2 was assessed in the chicken chorioallantoic membrane assay showing significant increase in blood vessel formation in response to ephrin B2, consistent with its reported role for endothelial cell activation. Thus, it appears that the need for multivalency for signaling can be met in the context of engineered fibrin that allows display of factors at high densities. Furthermore, because of the protection of fibrin-bound ephrin B2 from cleavage by proteases that are naturally complexed to this protein and terminate its activity, the prevention from such signal termination in the defined environment of the fibrin matrix could become useful for prolonging the functionality of the factor, and consequently prolonged activation of cellular binding partners.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

10. A method of attaching a bioactive factor to a matrix, comprising
recombinantly producing a biodomain peptide or protein comprising a bioactive factor and a transglutaminase substrate domain; and
exposing the matrix to a transglutaminase to covalently couple the bidomain peptide or protein to the matrix and crosslink the matrix.

11. The method of claim 10 wherein the matrix comprises fibrin.

13. The method of claim 10 wherein the transglutaminase substrate domain is a Factor XIIIa substrate domain and the transglutaminase is Factor XIIIa.

14. The method of claim 13 wherein the Factor XIIIa substrate comprises an amino acid sequence is selected from the group consisting of SEQ ID NO: 12, SEQ ID NO: 13, SEQ ID NO: 14, and SEQ ID NO: 15, and a combination or bioactive peptide fragment thereof.

15. The method of claim 14 wherein the Factor XIIIa substrate comprises an amino acid sequence of SEQ ID NO: 15.

16. The method of claim 10 wherein the bioactive factor is a polypeptide growth factor.

17. The method of claim 10 wherein the bioactive factor is selected from the group consisting of VEGF, growth factors from the TGF-β superfamily, PDGF, growth hormone, IGF, and ephrin.

18. The method claim 10 wherein the bioactive factor contains an acid sequence selected from the group consisting of SEQ ID NO:1, SEQ ID NO:2, SEQ ID NO:3, SEQ ID NO:4, SEQ ID NO:5, SEQ ID NO:6, TGF-β1, BMP 2; VEGF₁₂₁, PDGF AB, and PTH, and a combination or bioactive peptide fragment thereof.


SEQUENCE LISTING (33 sequences)

SEQ ID NO: 1 - protein, 5 aa, artificial sequence
  receptor-binding domain of laminin B1 chain
YIGSR

SEQ ID NO: 2 - protein, 5 aa, artificial sequence
  receptor-binding domain of laminin A chain
LRGDN

SEQ ID NO: 3 - protein, 5 aa, artificial sequence
  receptor-binding domain of laminin B1 chain
PDGSR

SEQ ID NO: 4 - protein, 5 aa, artificial sequence
  recognition sequence of the laminin A chain
IKVAV

SEQ ID NO: 5 - protein, 10 aa, artificial sequence
  recognition sequence of the laminin B2 chain
RNIAEIIKDI

SEQ ID NO: 6 - protein, 4 aa, artificial sequence
  collagen peptide that binds to the alpha 2 beta 1 integrin
DGEA

SEQ ID NO: 7 - protein, 6 aa, artificial sequence
  heparin-binding sequence from fibronectin
PRRARV

SEQ ID NO: 8 - protein, 16 aa, artificial sequence
  peptide with glutamine at the transglutaminase coupling site and an active peptide that mimics the crosslinking site in the gamma chain of fibrinogen
YRGDTIGEGQQHHLGG

SEQ ID NO: 9 - protein, 9 aa, artificial sequence
  peptide that mimics the lysine coupling site in the gamma chain of fibrinogen with an active peptide sequence
LRGDGAKDV

SEQ ID NO: 10 - protein, 8 aa, artificial sequence
  random coupling polylysine peptide attached to active peptide
LRGKKKKG

SEQ ID NO: 11 - protein, 12 aa, artificial sequence
  peptide that mimics the crosslinking site in alpha 2- plasmin inhibitor with an active peptide sequence
LNQEQVSPLRGD

SEQ ID NO: 12 - protein, 16 aa, artificial sequence
  peptide with glutamine at the transglutaminase coupling site in the chain of fibrinogen
YRGDTIGEGQQHHLGG

SEQ ID NO: 13 - protein, 5 aa, artificial sequence
  peptide that mimics the lysine coupling site in the chain of fibrinogen
GAKDV

SEQ ID NO: 14 - protein, 4 aa, artificial sequence
  peptide with a polylysine at a random coupling site
KKKK

SEQ ID NO: 15 - protein, 8 aa, artificial sequence
  peptide that mimics the crosslinking site in alpha 2- plasmin inhibitor ( abbreviated TG)
NQEQVSPL

SEQ ID NO: 16 - protein, 15 aa, artificial sequence
  peptide that mimics the crosslinking site in alpha 2- plasmin inhibitor with active peptide sequence
LNQEQVSPLGYIGSR

SEQ ID NO: 17 - protein, 15 aa, artificial sequence
  peptide that mimics the crosslinking site in alpha 2- plasmin inhibitor with active peptide sequence
LNQEQVSPLDDGEAG

SEQ ID NO: 18 - protein, 16 aa, artificial sequence
  peptide that mimics the crosslinking site in alpha 2- plasmin inhibitor with active peptide sequence
LNQEQVSPLRAHAVSE

SEQ ID NO: 19 - protein, 17 aa, artificial sequence
  peptide that mimics the crosslinking site in alpha 2- plasmin inhibitor with active peptide sequence
LNQEQVSPRDIKVAVDG

SEQ ID NO: 20 - protein, 19 aa, artificial sequence
  peptide that mimics the crosslinking site in alpha 2- plasmin inhibitor with active peptide sequence
LNQEQVSPRNIAEIIKDIR

SEQ ID NO: 21 - DNA, 54 bp, Homo sapien
cgcggatccaatcaagaacaagtcagtccccttgcacccatggcagaaggagga

SEQ ID NO: 22 - DNA, 35 bp, Homo sapien
ggaattcctcaccgcctcggcttgtcacaattttc

SEQ ID NO: 23 - protein, 6 aa, artificial sequence
  thrombin cleavage site
GLVPRG

SEQ ID NO: 24 - DNA, 26 bp, artificial sequence
  1S DNA primer
cccggatcccgcagcgcaattgagaa

SEQ ID NO: 25 - DNA, 35 bp, artificial sequence
  2AS DNA primer
cccgaattcttattactgtgccctgctctccaacc

SEQ ID NO: 26 - DNA, 52 bp, artificial sequence
  3S DNA primer
cccggatccaatcaggaacaggtttctcctttggaagcaacccagatcacac

SEQ ID NO: 27 - protein, 1260 aa, Mus Musculus
  MISC_FEATURE (516)..(604): the sixth Ig-like domain of the cell adhesion molecule L1
MVVMLRYVWPLLLCSPCLLIQIPDEYKGHHVLEPPVITEQSPRRLVVFPTDDISLKCEARGRPQVEFRWTKDGIHFKPKEELGVVVHEAPYSGSFTIEGNNSFAQRFQGIYRCYASNKLGTAMSHEIQLVAEGAPKWPKETVKPVEVEEGESVVLPCNPPPSAAPPRIYWMNSKIFDIKQDERVSMGQNGDLYFANVLTSDNHSDYICNAHFPGTRTIIQKEPIDLRVKPTNSMIDRKPRLLFPTNSSSRLVALQGQSLILECIAEGFPTPTIKWLHPSDPMPTDRVIYQNHNKTLQLLNVGEEDDGEYTCLAENSLGSARHAYYVTVEAAPYWLQKPQSHLYGPGETARLDCQVQGRPQPEITWRINGMSMETVNKDQKYRIEQGSLILSNVQPTDTMVTQCEARNQHGLLLANAYIYVVQLPARILTKDNQTYMAVEGSTAYLLCKAFGAPVPSVQWLDEEGTTVLQDERFFPYANGTLSIRDLQANDTGRYFCQAANDQNNVTILANLQVKEATQITQGPRSAIEKKGARVTFTCQASFDPSLQASITWRGDGRDLQERGDSDKYFIEDGKLVIQSLDYSDQGNYSCVASTELDEVESRAQLLVVGSPGPVPHLELSDRHLLKQSQVHLSWSPAEDHNSPIEKYDIEFEDKEMAPEKWFSLGKVPGNQTSTTLKLSPYVHYTFRVTAINKYGPGEPSPVSESVVTPEAAPEKNPVDVRGEGNETNNMVITWKPLRWMDWNAPQIQYRVQWRPQGKQETWRKQTVSDPFLVVSNTSTFVPYEIKVQAVNNQGKGPEPQVTIGYSGEDYPQVSPELEDITIFNSSTVLVRWRPVDLAQVKGHLKGYNVTYWWKGSQRKHSKRHIHKSHIVVPANTTSAILSGLRPYSSYHVEVQAFNGRGLGPASEWTFSTPEGVPGHPEALHLECQSDTSLLLHWQPPLSHNGVLTGYLLSYHPVEGESKEQLFFNLSDPELRTHNLTNLNPDLQYRFQLQATTQQGGPGEAIVREGGTMALFGKPDFGNISATAGENYSVVSWVPRKGQCNFRFHILFKALPEGKVSPDHQPQPQYVSYNQSSYTQWNLQPDTKYEIHLIKEKVLLHHLDVKTNGTGPVRVSTTGSFASEGWFIAFVSAIILLLLILLILCFIKRSKGGKYSVKDKEDTQVDSEARPMKDETFGEYRSLESDNEEKAFGSSQPSLNGDIKPLGSDDSLADYGGSVDVQFNEDGSFIGQYSGKKEKEAAGGNDSSGATSPINPAVALE

SEQ ID NO: 28 - protein, 42 aa, artificial sequence
  peptide, TGPTH,  synthesized using a Factor XIIIa substrate sequence (TG) and the first 34 amino acids of the parathyroid hormone (PTH)
NQEQVSPLSVSEIQLMHNLGKHLNSMERVEWLRKKLQDVHNF

SEQ ID NO: 29 - DNA, 51 bp, artificial sequence
  a sense DNA primer
cgcggatccaatcaagaacaagtcagtccccttaagtccatcgttttagac

SEQ ID NO: 30 - DNA, 44 bp, artificial sequence
  an antisense DNA primer
agtcacgatgcggccgcgcagcattctgaacccagtatactgga

SEQ ID NO: 31 - DNA, 34 bp, artificial sequence
  a sense DNA primer encoding part of the Factor XIIIa substrate and a custom Ndel site
ggaattccatatgaatcaagaacaagtcagtccc

SEQ ID NO: 32 - DNA, 30 bp, artificial sequence
  an antisense DNA primer with a stop codon and BamHI site
cgcggatcctcattctgaacccagtatact

SEQ ID NO: 33 - protein, 9 aa, artificial sequence
  a mutated ephrin B2 extracellular domain peptide motif
MNQEQVSPL